(19) United States
(12) Patent Application Publication
(10) Pub. No.: US 2006/0258991 A1
(43) Pub. Date: Nov. 16, 2006

(54) Portable needle uncapping and recapping device

(76) Inventor: Edward D. Lin, Osprey, FL (US)

Correspondence Address: RAY L. WEBER;RENNER, KENNER, GRIEVE, BOBAK, TAYLOR & WEBER, 400 First National Tower, Akron, OH, 44308, US

(21) Appl. No.: 11/118,901

(22) Filed: Apr. 29, 2005

(51) Int. Cl. A61M 5/00

(52) U.S. Cl. 604/263; 604/192

(57) ABSTRACT

A compact, substantially planar needlestick prevention safety device has a discoid shield-like portion, divided into an upper and lower half via a living hinge, and a pair of squeezable handles extending laterally therefrom. A central beveled aperture with outwardly extending radial slits accommodates within it a wide range of needle cap sizes. When the handles are squeezed by hand, the upper and lower halves of the disc fold toward each other along the living hinge and impart a variable and easily controllable amount of clamping force at the central aperture, thereby securely gripping the needle cap for uncapping or recapping purposes. The device provides a very high margin of safety during recapping, fits easily in the coat pocket for ease of accessibility, and is reusable and sterilizable by any sterilization method.

### TECHNICAL FIELD

The invention herein relates to medical devices and, more specifically, to a safety and protective device that prevents accidental needlestick injuries to healthcare workers.

### BACKGROUND ART

Accidental needle puncture injuries, known in the healthcare profession as “needlestick injuries” are a common and ever present risk of exposure to blood borne pathogens including HIV and hepatitis viruses.

In the early 1990s, 1.2 million accidental needle puncture injuries occurred annually in the US, costing $750 million in injury testing alone. Most occurred during the recapping process when a contaminated needle has to be returned to its cap or sheath via an aperture that is barely 5 to 10 mm in diameter. In the time-pressured environment of patient care, it is not too difficult, and only a matter of time before a healthcare worker makes an error judging the distance between the tip of the needle and needle cap aperture. The unfortunate consequence could be an accidental needlestick with a contaminated needle. In an attempt to bring down the incidence of such injuries, OSHA promulgated rules that forbid recapping unless an “engineered device” or safety device is used. A proliferation of safety products, fueled by OSHA mandates, have now helped to reduce the incidence to about 800,000 per year. These advances have been in the areas of universal deployment of sharps containers, safety syringes and needles, recapping devices, and needleless systems for drug delivery.

Although many safety needles and safety syringes provide ingeniously devised methods for either self-blunting or retraction, they all suffer from one major drawback of high cost. Costing as much as ten times that of the standard needle and syringe, they are, as a practical matter, unaffordable by most healthcare facilities. They also increase the amount of medical waste, which is quite expensive to dispose.

Even though one sharps container is typically placed on the wall of every semi-private patient room in the US, and at every nursing station, opportunities for accidental needlestick still occur. Picture for instance, the nurse just finishing an injection behind the drawn curtain around the patient's bed. Before she can get to the sharps container on the other side of the curtain, a colleague or a visitor or even the patient can bump into the nurse thus causing her to stick herself accidentally with the contaminate needle. The ideal way to contain the risk is immediate recapping with a simple to use and easy to carry safety device. Although portable recapping devices do exist, they are typically bulky and awkward to carry, thereby effectively limiting their wide acceptance and use. Since the primary objective of this invention is to disclose a simple to use and easy to carry safety device, irrelevant prior art pertaining to improvements in retractable needles, needle covers and safety syringes will not be discussed here.

In the area of portable recapping devices, all of the prior art suffers from a variety of drawbacks which have limited their wide acceptance.

U.S. Pat. No. 4,610,667 to Pedicano et al. discloses a disposable large-diameter safety needle sheath consisting of a tubular sleeve with a large funnel at one end serving as a guiding shield, the funnel being further provided with a lid. This device is meant to be a substitute needle cap to contain a used needle. It employs excessive material, is too bulky and cumbersome to use, and completely fails to take into consideration the reality of the healthcare work place.

U.S. Pat. No. 4,717,386 to Simmons discloses a plate like hand-held device with a central sheath holding structure for engaging a needle cap. This device has a long handle, thus increasing its bulk to an unacceptable degree.

U.S. Pat. No. 4,781,697 to Slaughter discloses a small protective shield with a central aperture that must first be applied to a needle cap to provide a slightly larger diameter of shielding. It does not have any provision for accepting caps of diameter other than its central aperture and the extra time and step needed to put this shield in place before use are major disadvantages.

U.S. Pat. No. 4,919,656 to Bracker et al. discloses a rigid disk with a central aperture surrounded by radially projecting teeth that grip onto a needle cap thrusted through the aperture from either side. No provisions exist to ease the insertion of the needle cap into the aperture, nor to accept a non-tapered needle cap or a wide range of needle cap sizes.

U.S. Pat. No. 4,981,476 to Aichlmayr and Enslow discloses a portable recapping device comprising a funnel-like shield attached to a resilient specially shaped metal ribbon. Although capable of accommodating a variety of needle cap sizes, this invention, like all other known portable shields, shares a fundamental design disadvantage: it has a shield and a handle portion that are axially perpendicular to each other. If the funnel-like shield is small in diameter, it offers little additional protection. If the shield is large in diameter, which it should be in order to provide adequate protection, then the handle, being at a cross axis, results in a disadvantageously large bulk or overall size.

A bulky portable device does not fit easily into the lab coat pocket and is therefore unlikely to be at hand when the need for its use arises. All these designs fail to address the real-life needs of the healthcare worker. Pocket space is a premium and is reserved for what is easy to carry around and used often. Bulky devices take up too much valuable space in the coat pocket and will end up being rejected by the healthcare worker.

None of the prior art provides a device that is universally compatible with any medical or dental needle, phlebotomy needle or intravenous catheter, with tapered or non-tapered cap, that is also easy to use, effective and reliable in function, compact in form factor, reusable and economical in cost. Satisfying all of these factors encourages use and compliance and provides broad protection.

### SUMMARY OF THE INVENTION

In light of the foregoing, it is a first aspect of the invention to provide a novel and effective portable recapper that meets the above criteria and provides a large safety shield form factor that is uniquely in the same axial plane as the safety handle, and thus of exceptionally compact form for fitting easily into a coat pocket.

A further aspect of the invention is to provide means for holding a cap in a convenient position for receiving the needle after use, while minimizing risk of needle stick injury to a user when reinserting the needle into the cap.

It is yet another aspect of the invention to provide a medically useful device that provides a substantial surface for imprinting with useful indicia of relevant information for either education or medical advertising purposes.

The foregoing and other aspects of the invention that will become apparent as the detailed description proceeds are achieved by a pair of handles joined at one end by a discoid shield having a variable diameter central aperture and integrally hinged together at the junction of the upper and lower halves for pivotal movement between open and closed positions. The central aperture can additionally have optional radial slits that will further accommodate a variety of larger needle cap sizes and hold them in place by friction without compressive force on the handles. The device is held in one hand while the needle cap is introduced through the central aperture. Squeezing the handles cause the upper and lower halves of the shield to tilt toward each other, thus progressively reducing the aperture size from its neutral position and at the same time producing an increasing clamping force upon the needle cap. This results in a secure grip upon the needle cap and facilitates the uncapping of the needle. Once the needle is uncapped, the device can be set upon any substantially horizontal surface and the cap will remain held in the central aperture at approximately a 45-degree angle. This not only prevents the setup from rolling about or collapsing, but provides an option to recap the needle single-handedly upon completion of the procedure. Optionally, when the procedure is completed the user can pick up the device, squeeze the handles to securely grip the cap, and recap the needle with all parts of the user's hand far remote from the needle cap and needle tip.

### BRIEF DESCRIPTION OF DRAWINGS

For a complete understanding of the objects, techniques and structure of the invention, reference should be made to the following detailed description and accompanying drawings wherein:

FIG. 1 is a perspective drawing showing a side electronic view of the invention;

FIG. 2 is a cross sectional view of FIG. 1 taken along line 2-2;

FIG. 3 is a cross sectional view of FIG. 1 taken along line 3-3;

FIG. 4 is a cross sectional view of FIG. 1 taken along line 4-4; and

FIG. 5 is a cross sectional view of FIG. 1 taken along line 4-4 under actual use condition with a needle assembly through its central aperture.

### DETAILED DESCRIPTION OF PREFERRED EMBODIMENT FOR CARRYING OUT THE INVENTION

Other than in the reference to specific prior art, throughout this disclosure, including the claims, the term needle shall be deemed to include any sharp object, including, without limitation, not just medical and dental needles, but also intravenous catheters, phlebotomy sets, spinal, epidural and biopsy needles and trocars. Similarly, the term needle cap shall include the caps and sheaths for all such sharp or pointed devices.

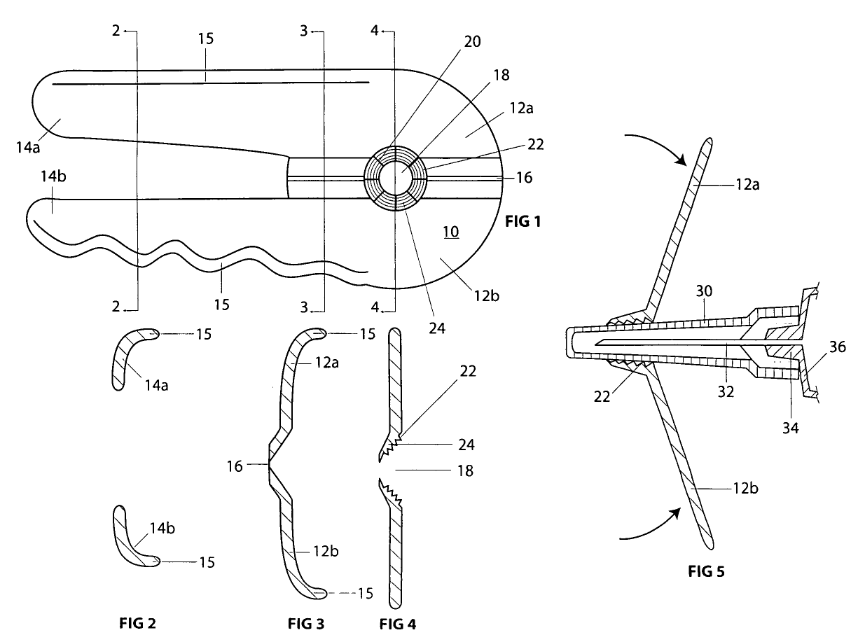

Referring now to FIG. 1, it can be seen that a portable needle uncapper and recapper according to the invention is designated by the numeral 10. The device 10 comprises two discoid upper and lower semicircular halves 12a and 12b joined together by a horizontal living hinge 16. Emanating from the same side of each of the upper 12a and lower 12b halves are two lateral handles 14a and 14b. When squeezed in the hand, the handles bring the two discoid halves toward each other, flexing along the living hinge 16, in a manner similar to partially closing the two halves of a hard-cover book. This is a mechanism and axis of movement distinctly different from a standard pair of pliers.

In the center of the discoid portion is an aperture or bore 18 through which the widest portion of the smallest-diametered needle cap could be inserted and held by friction. To accommodate different sizes of needle caps, the central aperture has radial slits 20 emanating from and spaced uniformly around it, dividing a tapered countersunk peripheral zone into radial tabs 24. When a needle cap larger than the central aperture is inserted, the radial tabs 24 yield with resistance, thereby holding the needle cap in place. As an optional feature, gripping protuberances or teeth 22, which can be in the form of rings, random nubs or other texturized surface structures, can be spaced about the beveled peripheral zone surrounding the central aperture. These will aid in gripping and securing the needle cap for uncapping or recapping.

Because the device is substantially planar, squeezing the handles very hard may be uncomfortable. Therefore, the handles 14a and 14b can have ergonomic comfort ridges or flanges 15 to diffuse pressure against the hand and fingers when the device handles are squeezed. FIG. 1 shows an optional embodiment in which the handles are ergonomically modified for a right-handed person. In this embodiment, handle 14b has finger indents or scallops for the left hand. The device is secured in the left hand while the right hand holds the needle-syringe assembly for uncapping, recapping or injection. The device can easily be rendered suitable for both left and right-handed users by eliminating the finger indents or scallops in the lower handle 14b so that the lower handle is a mirror image of upper handle 14a.

FIG. 2 is a cross sectional view of FIG. 1 taken along line 2-2 showing the handles with ergonomic pressure diffusing comfort flanges 15. As shown by FIGS. 2-4, the flange terminates as it leaves the handle portion. It can optionally extend around the periphery of the discoid halves, ending in the vicinity of the living hinge. Such extensions of the flange can provide additional protective function in stopping a grossly misdirected needle.

FIG. 3 is a cross sectional view of FIG. 1 taken along line 3-3. Here, the offset relationship of the shield halves 12a and 12b in relation to the living hinge 16, joining them in a horizontal axis can be seen. Any pressure applied to the handle immediately causes the shield halves to move toward each other in a closing manner. In the preferred embodiment, the entire device is made of a single piece of injection molded plastic such as polystyrene, and the living hinge consists of a suitably thin membrane of plastic material, capable of withstanding flexing under pressure for innumerable cycles. However, it should be evident to those skilled in the art that other material could be substituted and that other hinge mechanisms can be used without departing from the scope and spirit of this invention. For example, instead of plastic, metal sheet can be used, and small spring-loaded metal hinges can be deployed to each side of the central aperture.

In FIG. 4, which is a cross sectional view of FIG. 1 taken along line 4-4 of FIG. 1, it can be seen that the central aperture in the discoid shield is provided with a texturized biting surface which in this instance comprises ridges of concentric rings. The concentric and circumferential strip around the central aperture is countersunk to further facilitate both the insertion of the needle cap and the grasping action when the handles are squeezed.

With reference to FIG. 5, it can be seen how a needle cap 30 is gripped within the central aperture 18. A needle 32 is shown attached to a syringe 36, which is only partially shown. This gripping position is attained by squeezing the handles 14 which causes the upper and lower discoid shields 12a and 12b to tilt towards each other in directions shown by the two arrows, in a manner akin to closing the two halves of a hardcover book. As the two discoid halves close upon each other they exert a compressing and gripping force around the central aperture sufficient to grip and secure an element such as needle cap 30 interposed therein. During the recapping process, the V-shaped halves serve as a funnel-like shield to further channel any misdirected needle towards the central portion.

As shown in the drawings, the safety device 10 generally comprises a discoid shield 12 connected to an upper handle 14a and a lower handle 14b, which are designed for both left and right-handed use. The discoid shield 12 has a central aperture or bore 18 for receiving a needle cap 30. Aperture 18 is surrounded by a widely beveled circular strip with thickness ranging from approximately 3 to 5 mm, which is the full thickness of the discoid shield, at the outermost periphery, down to 0.5 to 1 mm in thickness at its innermost periphery. This tapering in thickness, along with radial slits 20, radial tabs 24 and gripping surface structures 22 helps to grip and retain needle caps within the central aperture.

As shown in FIG. 5, as the handles are squeezed, the discoid halves 12a and 12b tilt toward each other, simultaneously narrowing the central aperture and exerting a compression force upon the needle cap 30 that has been inserted into the central aperture.

To enhance the grip on needle cap 30, front plate 12 is countersunk with a surface texturized for gripping. The entire safety device is integrally formed of a piece of predominantly rigid material such as plastic, and be amenable to all forms of chemical gas or thermal sterilization.

The protective device 10 is used as follows. To begin, a user holds the needle-syringe assembly in one hand and the protective device 10 in the other. The user inserts the needle cap through the central aperture 18 and squeezes the handles 14a and 14b together to grip the cap. The user then pulls the syringe away from the device and the needle-syringe assembly becomes uncapped.

Preferably, the user then places the safety device on the table so that the entire device with the cap in its central aperture rests at an approximate 45 degree angle, thereby maintaining the cap open side up for later receiving the needle.

After using the syringe, the user reinserts the needle into the cap. This requires the use of only one hand, the hand holding the syringe, if the protective device holds the cap ready as noted above. In this way, the user's other hand may be kept away from the bare needle in order to completely eliminate the risk of a needle stick injury. Alternatively, some users may prefer to hold the safety device in one hand while reinserting the needle with the other. In that event, the hand holding the handles is remote from the needle cap, with virtually no risk of needle stick injury.

After the needle is securely engaged in the cap, the user releases the handles to release the cap and removes the syringe along with the recapped needle from the safety device.

A healthcare professional, for example, a plastic surgeon, an emergency physician or a dentist, may have occasion to administer medicine repeatedly to the same patient during a single procedure. Under such circumstances, it is unnecessary to change needles. During such a procedure, the safety device 10 conveniently holds the protective cap in an upright position by resting the safety device on a flat surface such as a surgical tray, so that the user can reinsert the needle into the cap between uses.

In summary, this invention discloses a unique and novel portable needle uncapper and recapper that is compact to carry, easy to use and effective in working with a wide range of needle cap sizes. The essentially planar structure combines a variably sized central aperture with a living hinge construction to provide a highly controllable and secure gripping mechanism for uncapping and recapping, and additionally eliminates the dual-axes bulkiness that is common to other portable needlestick prevention devices. In providing all the desirable characteristics that healthcare workers need in a portable safety device, this invention enables and encourages greater adoption of safety practices and help to reduce further the incidence of accidental needlestick injuries.

Having illustrated and described the principles of this invention in a preferred embodiment thereof, it should be readily apparent to those skilled in the art that the invention can be modified in arrangement and detail without departing from such principles. Accordingly, for an appreciation of the true scope of the invention, reference should be made to the accompanying claims.

What is claimed is:

1. A capping and uncapping device for needled medical implements, comprising:
a structure having first and second sections;
a hinge interconnecting said sections;
a bore passing through said hinge, said bore configured to receive a cap of a needled medical implement.

2. The capping and uncapping device as recited in claim 1, wherein said bore is tapered.

3. The capping and uncapping device as recited in claim 1, wherein said bore has a sidewall having radial slits therein, defining radial tabs therebetween.

4. The capping and uncapping device as recited in claim 1, wherein said bore sidewall is textured, having gripping protuberances extending therefrom.

5. The capping and uncapping device as recited in claim 1, wherein said structure comprises a pair of opposed handles, said hinge interconnecting said handles, and wherein said bore is substantially normal to a plane containing said pair of handles.

6. The capping and uncapping device as recited in claim 5, wherein said hinge is a living hinge of continuous thin flexible material.

7. The capping and uncapping device as recited in claim 6, wherein said bore has a sidewall having gripping protuberances extending therefrom.

8. The capping and uncapping device as recited in claim 7, wherein said bore sidewall has radial slits defining radial tabs therebetween.

9. The capping and uncapping device as recited in claim 8, wherein at least one of said handles has a flange along an outer edge thereof.

10. The capping and uncapping device for needled medical implements, wherein said flange is scalloped.

11. A capping and uncapping device for needled medical implements, comprising:
a discoid body of two opposing halves interconnected by a hinge;
a bore passing through said hinge; and
a pair of handles, one connected to each of said opposing halves of said discoid body and extending therefrom.

12. The capping and uncapping device as recited in claim 11, wherein said bore is substantially normal to said discoid body.

13. The capping and uncapping device as recited in claim 12, wherein said bore has a sidewall having an array of teeth extending inwardly therefrom.

14. The capping and uncapping device as recited in claim 13, wherein said sidewall is divided into separated sections.

15. The capping and uncapping device as recited in claim 14, wherein said bore is tapered inwardly of said discoid body.

16. The capping and uncapping device as recited in claim 15, wherein said sidewall of said bore is textured.
